(19) United States
(12) Patent Application Publication
(10) Pub. No.: US 2025/0248645 A1
(43) Pub. Date: Aug. 7, 2025

(54) PELVIC ORGAN PROLAPSE PRESSURE MEASURING DEVICE AND APPLICATION THEREOF

(71) Applicant: Cancer Hospital Chinese Academy of Medical Sciences, Beijing (CN)

(72) Inventors: Lin DENG, Beijing (CN); Bojun WANG, Beijing (CN)

(73) Assignee: Cancer Hospital Chinese Academy of Medical Sciences, Beijing (CN)

(21) Appl. No.: 19/015,695

(22) Filed: Jan. 10, 2025

(30) Foreign Application Priority Data
Feb. 5, 2024 (CN) 202410168071.6

(51) Int. Cl. A61B 5/00; A61B 5/03

(52) U.S. Cl. CPC A61B 5/4337; A61B 5/0004; A61B 5/036; A61B 2562/0247

(57) ABSTRACT

A pelvic organ prolapse pressure measuring device and an application thereof are provided. The pelvic organ prolapse pressure measuring device includes a pelvic organ prolapse pressure sensing system and a data acquisition system, the prolapse pressure sensing system includes a support body and pressure sensors which are distributed outside the support body. The pelvic organ prolapse pressure measuring device is applied to the pressure detection around the cavity. The above-mentioned pelvic organ prolapse pressure measuring device is adopted, which can objectively and accurately judge the abnormal stress relationship inside the prolapsed organ, and accurately evaluate the weak area of the pelvic floor muscle fascia. Its operation method is simple, and its cost is low.

### CROSS REFERENCE TO THE RELATED APPLICATIONS

This application is based upon and claims priority to Chinese Patent Application No. 202410168071.6, filed on Feb. 5, 2024, the entire contents of which are incorporated herein by reference.

### TECHNICAL FIELD

The invention relates to the technical field of gynecology and obstetrics and surgery, particularly, a pelvic organ prolapse (POP) pressure measuring device and an application thereof.

### BACKGROUND

Pelvic floor dysfunction disease (PFD) is a common chronic disease, the incidence rate of women in China is 25%, especially in postpartum women, middle-aged and elderly women, 50% of postmenopausal women have different degrees of pelvic floor dysfunction. However, at present, the diagnosis of pelvic floor dysfunction mainly depends on the gynecological examination of clinicians or the application of POP-Q score based on the relative position measurement of pelvic floor organs. The evaluation criteria are not objective and accurate enough, which differentiates the evaluation accuracy rate, therefore the views are different, and it is difficult to form a consensus. Although pelvic floor ultrasound and MRI have certain implications for pelvic floor dysfunction in recent years, there are also the following deficiencies:
(1) Objective and accurate diagnostic methods are lacked. The weak area of pelvic floor muscle fascia cannot be accurately evaluated, the abnormal stress relationship within the prolapsed organ cannot be objectively and accurately judged, it is suitable for severe patients with significant anatomical abnormalities.
(2) The operation method is complicated. It not only requires the operator to have rich clinical experience, but also requires the patient to simulate and reproduce the disease, resulting in high volatility, poor repeatability, and strong subjectivity of the measurement results.
(3) The remote and real-time detection cannot be fulfilled, and it is expensive. MRI equipment is heavy, and the examination is expensive. It can only be measured immediately and cannot be used frequently for a long time. The Intracavity coil is hard to be accepted, and the measurement range is incomplete. The image of the ultrasound examination has low resolution, which can only be measured immediately and takes a long time. It has high requirements for both operators and inspectors.

Therefore, it is urgent for a pressure measuring device, which can quickly, objectively, and accurately diagnose PFD, to unify the diagnostic criteria, which is easier for promotion and application.

### SUMMARY

The objective of the present invention is to provide a pelvic organ prolapse pressure measuring device and an application thereof. This device can objectively and accurately judge the abnormal stress relationship within the pelvic prolapsed organ, and accurately evaluate the weak area of the pelvic floor muscle fascia. The operation method is simple and cheap.

The invention proposes a pelvic organ prolapse pressure measuring device. The pelvic organ prolapse pressure measuring device includes a pelvic organ prolapse pressure sensing system and a data acquisition system. The pelvic organ prolapse pressure sensing system includes a support body, and pressure sensors which distributed outside of the support body.

Preferably, the data acquisition system includes a power source, a data collector, a data receiver, and a data transmitter arranged inside the support body.

Preferably, the data collector is electrically connected to the pressure sensor, the data collector collects an analog volume of a power signal from a measured unit of the sensor, and the data transmitter sends it to a server workstation according to a communication protocol. After the data format conversion and data structure processing, the server workstation performs data analysis. At the same time, the data receiver receives an instruction of the server workstation according to the communication protocol for data collection and manipulation.

Preferably, one side of the support body is spirally connected to a gripper, the gripper has a spiral connector, one side of the spiral connector is connected to an elastomer, one side of the elastomer is provided with an elastic plate. The other side of the spiral connector is connected to a handle, the handle includes a power switch and a detection controller. And one side of the handle is provided with a holding rod.

Preferably, the gripper's screwing end is provided with a sturdy pin. The interface of the support body is provided with a pin anchoring slot corresponding to the sturdy pin. The sturdy pin is stuck in the pin anchoring slot.

Preferably, the external side of the pressure sensor is coated with an isolator.

Preferably, the data transmitter and data receiver facilitate data communication in adherence to the communication protocol.

Preferably, the data transmitter uses transmission control protocol (TCP) data uninterrupted communication heartbeat packet, timely sends the power signal to the server workstation according to the communication protocol. The communication protocol is 5G mobile communication technology.

Preferably, this invention provides the application of the pelvic organ prolapse pressure measuring device in pressure detection around cavity.

Preferably, after sanitizing the pelvic organ prolapse pressure measuring device, insert it into the measurement site, select the real-time pressure measurement by the panel on the detection controller. Increase the pressure and perform the pressure measurement again. The pelvic organ prolapse pressure measuring device will send the collected power signal analog volume to the server workstation through the data communication system and the results are displayed on the monitor after real-time processing.

Therefore, the invention provides a pelvic organ prolapse pressure measuring device and the application thereof, which can objectively and accurately judge the abnormal internal force relationship of pelvic prolapsed organ, and accurately evaluate the weak area of pelvic floor muscle fascia. The operation method is simple, and the cost is low.

The following is a further detailed description of the technical scheme of the invention through figures and an embodiment.

### BRIEF DESCRIPTION OF THE DRAWINGS

FIG. 1 is an overall structure of the pelvic organ prolapse pressure measuring device in the invention.

FIG. 2 is a structural diagram of the support body of the data acquisition system in the invention.

FIG. 3 is a structural diagram of the gripper of the data acquisition system in the invention.

### THE APPENDED FIGURE REFERENCE SIGNS

1 support body; 2 flexible pressure sensor; 3 gripper; 31 power switch; 32 holding rod; 33 handle; 34 detection controller; 35 sturdy pin; 36 elastic plate; 37 spring; 4 isolator; 5 power source; 6 data collector; 7 data receiver; 8 data transmitter; 9 screwing slot of the spiral connector; 10 pin anchoring slot.

### DETAILED DESCRIPTION OF THE EMBODIMENTS

The following is a further explanation of the technical scheme of the invention through figures and an embodiment.

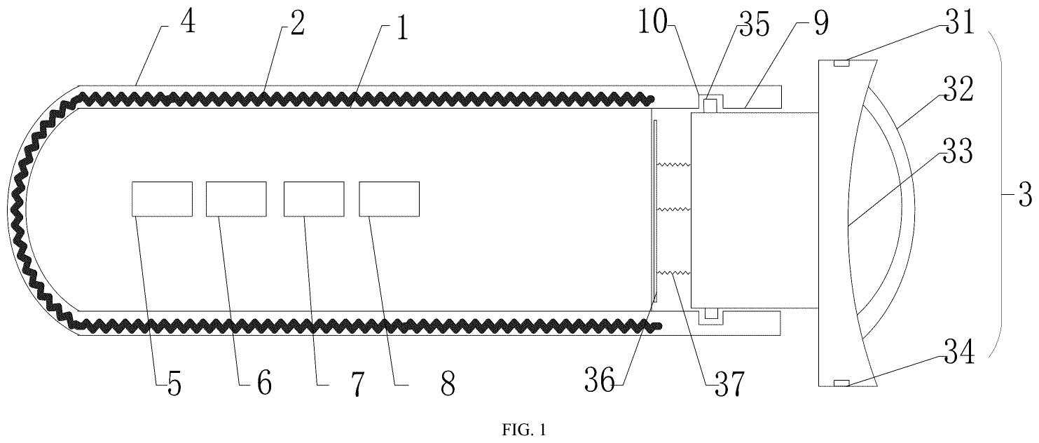

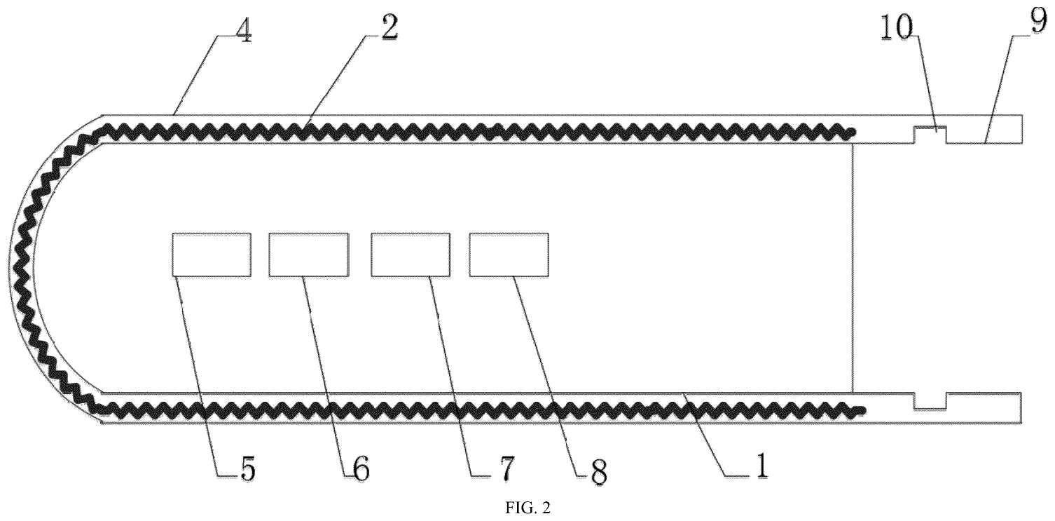

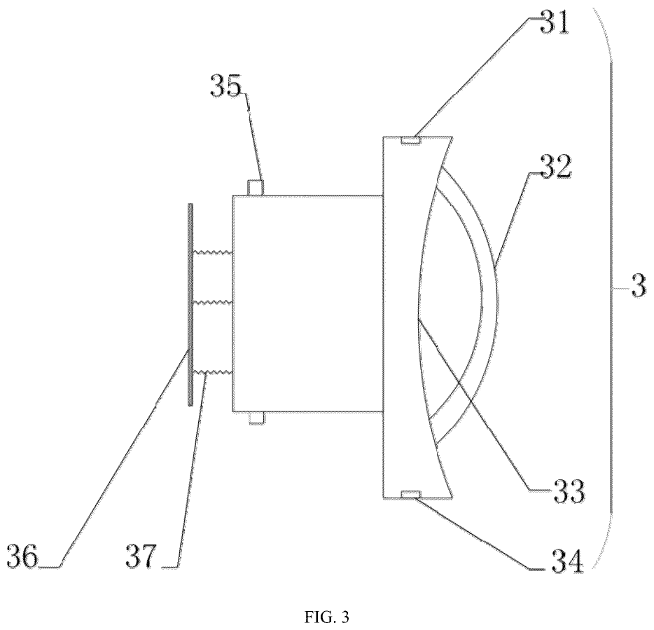

As shown in FIGS. 1-3, the invention provides a pelvic organ prolapse pressure measuring device. The pelvic organ prolapse pressure measuring device includes a pelvic organ prolapse pressure sensing system and a data acquisition system, the pelvic organ prolapse pressure sensing system includes a support body 1, and flexible pressure sensors 2 which are arrayed partition-wise on the outer wall of the support body. the flexible pressure sensor 2 is closely attached to the outer side of the support body 1, and the outer side of the flexible pressure sensor 2 is coated with an isolator 4 made of an ultra-thin flexible silicone material. a gripper 3 is spirally set on the side of the support body 1.

The data acquisition system includes a high-speed data collector (A/D card), a power source, a data transmitter, a data receiver, and a communication protocol. The high-speed data collector (A/D card) 6 is a multi-channel data acquisition card, which is connected to the flexible pressure sensor 2, and automatically collects the analog signal of the power signal from the measured unit of the sensor, the data transmitter 8 is sent to the server workstation according to the communication protocol, and the data is analyzed after the data format conversion and data structure processing, at the same time, the data receiver 7 receives the instruction of the server workstation for data collection and manipulation. The data communication between the data transmitter 8 and the data receiver 7 and the server workstation is realized through the communication protocol.

Among them, the support body 1 is fabricated from flexible and micro-deformable silicone gel, exhibiting both waterproof and insulation characteristics. The size of the support body 1 is set to the system model according to the detection object; one end of the supporting body 1 is spirally connected to the gripper 3, the other end of the supporting body 1 is hemispherical and the middle is cylindrical. The flexible pressure sensor 2 is coated on the outer side of the support body 1 in the array type. The flexible pressure sensor 2 performs pressure detection and diagnosis according to the specified partition. The outer side of the flexible pressure sensor 2 is covered with an isolator 4 made of an ultra-thin flexible silicone material. The inner side of the support body 1 is provided with a power supply 5, a data collector 6, a data receiver 7, and a data transmitter 8. The support body 1, the flexible pressure sensor 2, the gripper 3, the isolator 4, the power supply 5, the data collector 6, the data receiver 7, and the data transmitter 8 are all sterile isolated.

The connection between the support body 1 and the gripper 3 is provided with a screwing slot of the spiral connector 9 and a pin anchoring slot 10 to ensure that the support body 1 and the gripper 3 can be connected securely. The gripper 3 includes a spiral connector, the outer wall of the spiral connector is provided with corresponding threads which match the screwing slot of the spiral connector 9. One end of the spiral connector, which screwing into the support body 1, is provided with a spring 37. One end of the spring 37 is fixedly connected to an elastic plate 36. The end of the spiral connector near the support body 1 is also provided with a sturdy pin 35 corresponding to the pin anchoring slot 10. The other end of the spiral connector is provided with a handle 33 which equipped with a power switch 31 and a detection controller 34. The detection controller 34 controls the pressure detection and diagnosis according to the partition. One end of the handle 33 is provided with a gripper 32. The setting of the spring 37 keeps the elasticity between the support body 1 and the spiral connector and enhances the comfort during the detection process.

When using the gripper 3, it is screwed clockwise along the screwing slot of the spiral connector 9 into the interface of the support body 1. The sturdy pin 35 is stuck into the pin anchoring slot 10, ensuring a tight connection between body 1 and gripper 3.

The data transmitter 8 uses TCP data-based continuous communication heartbeat packets to send analog power signals to the server workstation according to the communication protocol at regular intervals, allowing the server workstation to know it is online and ensuring the validity of the connection.

The communication protocol adopted by data transmitter 8 and data receiver 7 are based on 5G mobile communication technology, offering high data rates, latency reduction, energy saving, cost, reduction and improves system capacity and large-scale device connectivity. The power source is button batteries.

The pelvic organ prolapse pressure measuring device sends the analog power signals to the server workstation according to the communication protocol at scheduled times. The server workstation performs format conversion and data structured processing of the data before perform data analysis. The server workstation includes a hardware part and a software part. The hardware part includes the server, the monitor, and workstation power supply. The software part includes the database, the data conversion module, the data structure module, the filtering processing modules, and the fusion analysis module.

The server workstation first converts the format through the data conversion module, performs structured processing according to the format preset by the data structure module, and stores it in the database. Then, it applies the filtering tools from the filtering processing module to filter out influencing factors and eliminate the interference signal and noise. And subsequently, the fusion analysis module is used for statistical analysis, correlation analysis and correlation analysis. After carrying out fusion processing according to the relevant factors, discriminant analysis and decision-making suggestions are provided.

The pelvic organ prolapse pressure measuring device involved in the invention is divided into different models according to the size, which can be used for pressure detection in different populations. The application range includes but is not limited to gynecology, urology, and colorectal, and can be used for the pressure detection around all similar cavities. It is mainly used in gynecology to measure pelvic floor pressure through the vagina, as well as in urology to measure the pressure around the urethra such as urethral sphincter. It can also be used in colorectal surgery to measure the pressure around the rectum.

When using the pelvic organ prolapse pressure measuring device the pressure measuring device is disinfected and placed in the patient's vagina up to the cervix/vaginal fornix. Select real-time pressure measurement on the control panel and save data after the pressure measurement. Then, the patient is instructed to cough or increase abdominal pressure and detect the pressure again. The pelvic organ prolapse pressure measurement device sends the collected analog electrical signals to the server workstation via the data communication protocol and displays the results on the monitor after the real-time data processing.

Therefore, the invention provides the above-mentioned pelvic organ prolapse pressure measuring device and the application thereof. It can objectively and accurately judge the abnormal stress relationship inside the pelvic prolapsed organ, and accurately evaluate the weak area of the pelvic floor muscle fascia. The operation method is simple and cheap.

In the description of this instruction, the reference terms an embodiment example, concrete example, etc. are used to describe at least one embodiment or example of the invention that incorporates the specific features, structure, material, or characteristics described by the embodiment or example. In this instruction, the indicative expression of the above terms does not necessarily refer to the same embodiment or example. Furthermore, the specific features, structures, materials, or features described can be combined appropriately in any one or more embodiments or examples.

Finally, it should be noted that the above embodiment is only used to explain the technical scheme of the invention rather than to restrict it. Although the invention is described in detail concerning the better embodiment, ordinary technicians in this field should understand that they can still modify or replace the technical scheme of the invention, and these modifications or equivalent replacements cannot make the modified technical scheme out of the spirit and scope of the technical scheme of the invention.

What is claimed is:

1. A pelvic organ prolapse pressure measuring device, comprising: a pelvic organ prolapse pressure sensing system and a data acquisition system, wherein the pelvic organ prolapse pressure sensing system comprises a support body, and pressure sensors are distributed outside of the support body.

2. The pelvic organ prolapse pressure measuring device according to claim 1, wherein the data acquisition system comprises a power supply, a data collector, a data receiver, and a data transmitter arranged inside the support body.

3. The pelvic organ prolapse pressure measuring device according to claim 2, wherein the data collector is electrically connected to the pressure sensors, and the data collector collects analog power signals from a measured unit of each of the pressure sensors, the data transmitter sends the analog power signals to a server workstation according to a communication protocol; and after a data format conversion and data structure processing, a data analysis is performed, at the same time, the data receiver receives an instruction of the server workstation according to the communication protocol for a data acquisition and a data operation.

4. The pelvic organ prolapse pressure measuring device according to claim 3, wherein a side of the support body is spirally connected to a gripper, the gripper comprises a spiral connector, a first side of the spiral connector is connected to an elastomer, a side of the elastomer is provided with an elastic plate, a second side of the spiral connector is connected to a handle, the handle is provided with a power switch and a detection controller, and a side of the handle is provided with a holding rod.

5. The pelvic organ prolapse pressure measuring device according to claim 4, wherein there is a sturdy pin set on a screw-in end of the gripper and there is a corresponding pin anchoring slot set at an interface of the support body, and the sturdy pin is engaged in the corresponding pin anchoring slot.

6. The pelvic organ prolapse pressure measuring device according to claim 5, wherein an isolator is provided outside the pressure sensors.

7. The pelvic organ prolapse pressure measuring device according to claim 6, wherein the data transmitter and the data receiver communicate data through the communication protocol.

8. The pelvic organ prolapse pressure measuring device according to claim 7, wherein the data transmitter adopts a transmission control protocol (TCP) data uninterrupted communication heartbeat packet, the analog power signals are timely sent to the server workstation according to the communication protocol, and the communication protocol is a 5G mobile communication technology.
